(19) United States
(12) Patent Application Publication
(10) Pub. No.: US 2002/0155496 A1
(43) Pub. Date: Oct. 24, 2002

(54) Saturated and unsaturated abietane derivatives, derived conjugates and uses in a diagnostic composition, a reagent and a device

(76) Inventors: Marie Helene Charles, Condrieu (FR); Nadia Piga, Ecully (FR); Nicole Battail-Poirot, Lyon (FR); Laurent Veron, Lyon (FR); Thierry Delair, Echalas (FR); Bernard Mandrand, Villeurbanne (FR)

Correspondence Address: OLIFF & BERRIDGE, PLC, P.O. BOX 19928, ALEXANDRIA, VA, 22320, US

(21) Appl. No.: 09/771,554

(22) Filed: Jan. 30, 2001

Related U.S. Application Data
(63) Continuation-in-part of application No. PCT/FR99/01846, filed on Jul. 27, 1999.

(30) Foreign Application Priority Data
Jul. 31, 1998 (FR) 98 10084

(51) Int. Cl. G01N 33/53; C07C 31/22; C07C 271/24; C07C 265/10; C07C 255/31; C07C 233/23

(52) U.S. Cl. 435/7.1; 560/117; 558/429; 560/354; 564/188; 568/38

(57) ABSTRACT

The invention concerns a saturated or unsaturated abietane derivative of general formula (I) wherein: Z is selected among —COOR⁵, —CONR¹R², —COONR³R⁴, —COR⁶, —CON, —COOR⁵, —CHOHR⁷, —SR⁸, —OR⁸, —CN, —CNO, —CNS, —NCO, —NCS and —R¹R²CR⁹; wherein R¹, R², R³ and R⁴ represent a hydrogen atom, a C₁-C₁₀ alkyl, a C₆-C₂₀ aryl optionally substituted, a C₇-C₁₀ alkene, a C₁-C₁₀ alkyne, or an aminoacyl or peptidyl optionally substituted; or R¹ and R² or R³ and R⁴ together form a cycle or a heterocycle; R⁵ represents a hydrogen, a C₁-C₁₀ alkyl, a C₁-C₁₀ alkene, a C₁-C₁₀ alkyne, or an aryl optionally substituted into C₆-C₂₀; R⁶ represents a hydrogen, a halogen, a C₁-C₁₀ alkyl, a C₁-C₁₀ alkene, a C₁-C₁₀ alkyne, or an aryl optionally substituted into C₆-C₂₀; R⁷ represents a hydrogen, a C₁-C₁₀ alkyl, a C₁-C₁₀ alkene, or a C₁-C₁₀ alkyne; R⁸ represents a hydrogen, a C₁-C₁₀ alkyl, a C₁-C₁₀ alkene, or a C₁-C₁₀ alkyne; and R⁹ is selected among —CN, —CNO, —CNS, —NCO and —NCS. The invention also concerns a derived conjugate and the use of the derivative and the conjugate in a diagnostic composition, a reagent and a device.

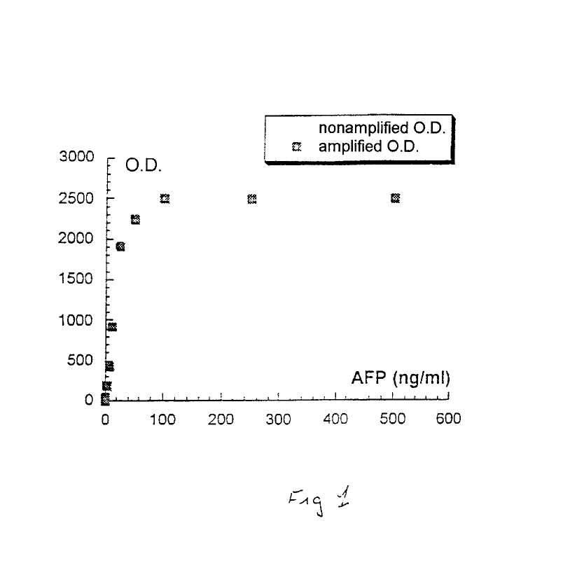

A subject matter of the present invention is novel abietane derivatives, novel abietane-derived conjugates and their uses. Compounds for chemoluminescent labelling comprising a biotin part, which are capable of attaching to streptavidin or avidin, and their drifts conjugates are known from U.S. Pat. No. 5 395 938 and a labelling technique which consists of the use of fluorescent microspheres which will interact with proteins, such as avidin or an immunoglobulin, is known from U.S. Pat. No. 5 132 242. The development of these techniques leads to the search for molecules capable of being used either as labels or to constitute a universal solid phase or for the production of monoclonal or polyclonal antibodies.

Acids and derivatives of abietic acid extracted from pine essences and resins and other resins, such as abietane, pimaric acid and isopimaric acid, used as solvent, for example, are known. Mention will be made of the extraction of compounds, such as dehydroabietinal, abietinal and abietinol, from Pinus silvestris resin taught in the article by E. N. Schmidt et al. in 1968 (CAS, vol. 70, No. 9, 35043p). More recently, abietane derivatives have been studied for their pharmacological properties, in particular as hypocholesterolemics intended for the treatment of arteriosclerosis, as taught, for example, in Patents DE-A-2 519 943, FR-A-2 282 872 and DE 2 521 088. Various processes for the synthesis of abietic acid derivatives have been provided in publications, such as that of H. Derek et al. in 1983 (J. Chem. Soc., Chem. Comm., 17, 939), relating to the decarboxylation of acids, or that of B.E. Cross et al. in 1981 (J. C. S. Perkin I, 3158), providing for the synthesis of lactones from 8β, 13β-tetrahydroabietic acid, or that of I. I. Bardyshev et al. in 1968 (CAS, vol. 69, No. 9, 362829), describing the preparation of abietic anhydride.

The Applicant company has unexpectedly discovered that abietane derivatives can be used in the field of diagnostic tests.

The novel derivatives comprise the basic abietane backbone, which corresponds to the formula below

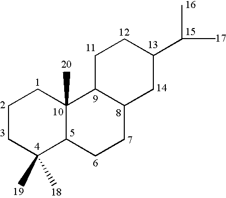

said molecule comprising, in the 18-position, a reactive functional group Z.

The novel abietane derivatives can be used in a large number of analyses. By way of example, they can be used directly or indirectly in the development of novel diagnostic tests; in the monitoring of infection, for example of a viral infection; in the monitoring and/or the assay of chemicals and in particular of potentially polluting products.

In diagnostic tests, they can be used either as labels or to form a universal solid phase or in the production of monoclonal or polyclonal antibodies which can be used for diagnostic purposes. As such, they can advantageously substitute for biotin in developing novel diagnostic tests. This is because they are chemical molecules which are not found in human beings, in particular in the serum, which means that they allow potential interactions with biological molecules to be avoided.

In the assay and/or the monitoring of chemicals, as the abietane derivatives according to the invention are relatively inert chemically, they can be used as labels in numerous tests for the assay of chemical compounds without disturbing the physical and/or chemical and/or physicochemical properties of the latter.

With regard to the synthesis, as they are monofunctional, they can be easily synthesized in a chemospecific fashion.

By way of example, a few applications of the novel abietane derivatives in immunological assay tests will now be expanded upon:

Use of said derivatives as labels in a diagnostic test:

The novel abietane derivatives can be used as labels in an immunoanalysis test, for example for the assay and/or the quantification of antibodies specific for an antigen in a biological sample by the competition technique. To this end, the antigen is attached to a solid phase, such as, for example, represented by a well of a microtitration plate. The biological sample to be assayed and a predetermined amount of an antibody conjugated to an abietane derivative are added to the solid phase and the formation of antigen/antibody conjugated to an abietane derivative complexes is detected using an antibody directed against the abietane derivative, said antibody being labeled by any appropriate label. Similarly, the abietane derivatives can be used as labels in a test for the assay and/or the quantification of an antigen in a biological sample by a competition technique. In this case, an antibody specific for the antigen to be assayed is attached to a solid phase and both the biological sample capable of comprising the desired antigen and a predetermined amount of an antigen derivative conjugated to an abietane derivative are added to the solid phase thus constituted. The formation of the antibody/antigen derivative conjugated to an abietane derivative complexes is subsequently demonstrated by the addition of an antibody directed against the abietane derivative, said antibody being labeled by any appropriate label. Likewise, the abietane derivatives can be used as labels in a sandwich technique in searching for an antigen or for an antibody in a sample. To this end, the antibody/antigen of the sample immune complex is demonstrated by complexing with a second antibody conjugated to an abietane derivative or by complexing with a second antigen conjugated to an abietane derivative and revelation is carried out using an antibody directed against the labeled abietane derivative, said antibody being labeled by any appropriate label.

The abovementioned abietane derivatives are coupled to a biological molecule, such as a protein chosen from antibodies, antigens and polypeptides, directly or indirectly. Indirectly, they are coupled via compounds having at least two identical or different reactive functional groups, such as spacer arms or natural or synthetic polymers defined in more detail below. The abietane derivatives, thus coupled, are used in a reagent for the diagnosis. Thus, another subject matter of the present invention is a reagent additionally comprising an abietane derivative coupled to a protein chosen from polypeptides, antigens and antibodies.

The invention also relates to the use of such a reagent in a diagnostic test and to a composition additionally comprising a reagent as defined above.

Use of the abietane derivatives in forming a universal solid phase:

By way of example, antibodies directed against an abietane derivative are immobilized on a solid phase or support, directly or indirectly, and a polypeptide or an oligonucleotide is modified by attachment at one of its ends of an abietane derivative. The antibody directed against the abietane derivative/abietane derivative-polypeptide complex or the antibody directed against the abietane derivative/abietane derivative-oligonucleotide complex is subsequently used according to conventional sandwich or competition techniques. The universal solid phase is composed of the solid phase/antibody directed against an abietane derivative assembly. Another subject matter of the present invention is thus a device comprising, in addition to the solid phase or support, a polyclonal or monoclonal antibody directed against an abietane derivative, said antibody being immobilized directly or indirectly on said solid phase or support. The antibody is preferably a monoclonal antibody obtained according to known techniques, by way of reference that described in Example 10.

The derivatives of the invention can be used for the assay and/or the monitoring of chemicals in a liquid medium, in particular an aqueous medium, by a sandwich technique or by a competition technique, as is described above for the diagnostic test.

A subject matter of the present invention is consequently novel saturated or unsaturated abietane derivatives corresponding to the generic formula (I):

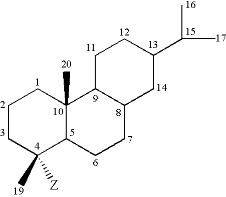

in which Z is chosen from the group consisting of —COOR⁵, —CONR¹R², —COONR³R⁴, —COR⁶, —COOR⁵, —CHOHR⁷, —SR⁸, —OR⁸, —CN, —CH₂NO, —CH₂NS, —NCO, —NCS or —R¹R²CR⁹;

in which R¹, R², R³ and R⁴, independently of one another, represent a hydrogen atom, an alkyl radical comprising from 1 to 10 carbon atoms, an aryl radical preferably comprising from 6 to 20 carbon atoms, which is optionally substituted; an alkene radical comprising from 7 to 10 carbon atoms; an alkyne radical comprising from 2 to 10 carbon atoms; or an optionally substituted aminoacyl or peptidyl radical, or R¹ and R² or R³ and R⁴ can together form a ring or a heterocycle; R⁵ represents a hydrogen atom, an alkyl radical comprising from 1 to 10 carbon atoms, an alkene radical comprising from 2 to 10 carbon atoms, an alkyne radical comprising from 2 to 10 carbon atoms or an optionally substituted aryl radical comprising from 6 to 20 carbon atoms; R⁶ represents a hydrogen atom, a halogen, an alkyl radical comprising from 1 to 10 carbon atoms, an alkene radical comprising from 2 to 10 carbon atoms, an alkyne radical comprising from 2 to 10 carbon atoms or an optionally substituted aryl radical comprising from 6 to 20 carbon atoms; R⁷ represents a hydrogen atom, an alkyl radical comprising from 1 to 10 carbon atoms, an alkene radical comprising from 2 to 10 carbon atoms or an alkyne radical comprising from 2 to 10 carbon atoms; R⁸ represents a hydrogen atom, an alkyl radical comprising from 1 to 10 carbon atoms, an alkene radical comprising from 1 to 10 carbon atoms or an alkyne radical comprising from 1 to 10 carbon atoms; and R⁹ is chosen from —CN, —CH₂NO, —CH₂NS, —NCO and —NCS;

provided that

(a) if said abietane derivative is a saturated derivative:

Z does not represent any one of the following radicals: —COOH, —NCO, —CONH₂, —CN, N-benzylamide, N-isopropylamide, N-cyclohexylamide, N-cyclopentylamide, N-α-phenylethylamide, N,N-dibenzylamide, N-methyl-N-cyclohexylamide, N-methyl-N-phenylamide, N-phenyl-N-benzylamide, anilide and —CONH[—(CH₂)ₘ—C₆H₅₋ₙX′ₙ], where m is equal to 0 or 1, n is equal to 1, 2 or 3, and X′ represents a halogen atom, a lower alkyl group,like methyl or ethyl group, a haloalkyl group, a hydroxyl group, a lower alkoxy group, like methoxy or ethoxy group, a nitro group, a carbonyl group, an alkoxycarbonyl group and a methyl group, and

(b) if said abietane derivative is an unsaturated derivative:

Z does not represent any one of the following radicals: —COOH, N-isopropylamide, N-methyl-N-cyclohexylamide, N-cyclohexylamide, N-decyl-amide, N-dodecylamide, N-pentadecylamide, N-allylamide, N,N-diallylamide, N-cycloheptylamide, N-cyclopentylamide, N-benzyl-amide, N-α-phenylethylamide, N-α-phenyl-propylamide, N,N-dibenzylamide, N-p-phenyl-ethylamide, N-ethyl-N-benzylamide, N-methyl-N-phenylamide, anilide and —CONH[—(CH₂) ₘ—C₆H₅₋ₙX′ₙ], where m is equal to 0 or 1, n is equal to 1, 2 or 3, and X′ represents a halogen atom, a lower alkyl group,like methyl ou ethyl group, a haloalkyl group, a hydroxyl group, a lower alkoxy group,like methoxy ou ethoxy group, a nitro group, a carbonyl group, an alkoxycarbonyl group and a methyl group,

if Z represents —COOR⁵, R⁵ represents neither H nor a methyl, ethyl or benzyl radical,

if Z represents —CONR¹R² and if one of R¹ and R² represents H, the other of R¹ and R² does not represent H, and if one of R¹ and R² represents the ethyl radical, the other of R¹ and R² does not represent the ethyl radical,

if Z represents —COR⁶ or —CHOHR⁷, R⁶ and R⁷ do not represent H.

The term “saturated abietane derivative” is understood to mean a derivative having the abovementioned abietane backbone in which the three rings and the side group in the 13-position do not comprise any unsaturation, independently of the definition of Z. The term “unsaturated derivative,” is understood to mean a derivative having the abietane backbone in which at least one of the three rings and/or the side group in the 13-position comprises an unsaturation. Mention may be made, as examples of unsaturated derivatives, of those for which the backbone is chosen from the abietic acid, dehydroabietic acid or neoabietic acid backbones, independently of the definition of Z.

The alkyl radicals mentioned above preferably comprise from 1 to 6 carbon atoms. They can be linear or branched. They are preferably linear radicals. An alkyl radical can be interrupted by one or more heteroatoms and/or can be substituted or unsubstituted.

The aryl radicals mentioned above preferably comprise from 6 to 14 carbon atoms. They can be cyclic or heterocyclic compounds which are optionally substituted, in particular by heteroatoms or groups of heteroatoms, such as nitro, sulfo and sulfonate groups.

Advantageously, —COONR³R⁴ represents an N-hydroxysuccinimide ester, —COR⁶ represents an acid chloride, —CONR¹R² represents an N-substituted amide group in which R¹ and R², independently of one another, represents a hydrogen atom or a polyethylene glycol radical and advantageously a tetra- or hexaethylene glycol radical or alternatively an optionally substituted peptidyl radical comprising from 2 to 6 aminoacyl residues. The peptidyl radical is preferably a glycyl-glycine radical and advantageously the glycyl-glycine radical is substituted by N-hydroxysuccinimide; —COORs preferably represents a polyethylene glycol ester and advantageously a tetra- or hexaethylene glycol ester.

Another subject matter of the present invention is novel abietane-derived conjugates which correspond to the general formula (II)

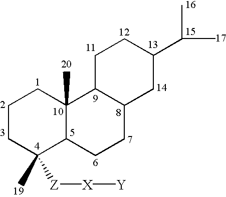

in which Z represents a radical as defined in the preceding formula (I); X represents a spacer arm chosen from an aliphatic chain (CH₂)n, in which n is an integer between 0 and 10 and preferably equal to 6, an ethylene glycol or a polyethylene glycol, preferably a tetra- or hexaethylene glycol, or an aminoacyl or peptidyl residue and in particular a peptide chain comprising from 2 to 10 amino acids; Y represents a polymer chosen from proteins, polypeptides, polynucleotides or oligonucleotides, and chemical polymers;

provided that, if said conjugate comprises an unsaturated abietane derivative, Y is not BSA.

The term “polymer” is understood to mean a molecule or a macromolecule composed of at least two monomer units.

Thus, a protein is a macromolecule, of natural origin or obtained by a synthetic route or by a genetic recombination technique, exhibiting an average molecular mass of approximately at least 200 daltons.

A polypeptide corresponds to a series of at least two amino acids, preferably of 2 to 20 amino acids, and its equivalents; said polypeptides being obtained by a chemical synthetic route and/or by fragmentation of a native protein using appropriate restriction enzymes and/or by genetic recombination. A polypeptide is said to be equivalent with respect to a reference polypeptide if it exhibits substantially the same properties and in particular the same antigenic, immunological, enzymological and/or molecular recognition properties. The following are in particular equivalent to a reference polypeptide: any polypeptide having a sequence in which at least one amino acid is substituted by an analogous amino acid, that is to say an amino acid which exhibits substantially the same physicochemical characteristics as a reference amino acid; any polypeptide exhibiting an equivalent peptide sequence, obtained by natural or induced variation of the reference polypeptide and/or of the nucleotide fragment coding for said polypeptide; a mimotope, any polypeptide in the sequence of which one or more amino acids of the L series are replaced by one or more amino acids of the D series and vice versa; any polypeptide into the sequence of which has been introduced a modification of the side chains of at least one amino acid, such as, for example, acetylation of the amine functional groups, carboxylation of the thiol functional groups or esterification of the carboxyl functional groups; polypeptide in the sequence of which one or more peptide bonds have been modified, such as, for example, carba, retro, inverso, retro-inverso or methyleneoxy bonds, and any polypeptide with at least one antigen recognized by an antibody directed against a reference peptide.

A polynucleotide or oligonucleotide corresponds to a series of at least two monomer units, in particular of at least five monomers and preferably of 5 to 22 monomers, advantageously of 18 to 22 monomers and preferably of 20 monomers, characterized by the informational sequence of the natural nucleic acids, capable of hybridizing under predetermined conditions to a nucleotide fragment, it being possible for the series to comprise monomers of different structures and to be obtained by a chemical synthetic route and/or by fragmentation of a natural nucleic acid and/or by genetic recombination. Thus, a monomer can be a nucleic acid natural nucleotide, the constituent components of which are a nitrogenous base, a sugar and a phosphate group, or a nucleotide modified in at least one of the three constituent components; by way of example, the modification can take place at the bases, generating modified bases, such as inosine, 5-methyldeoxycytidine, deoxyuridine, 5-dimethylaminodeoxyuridine, 2,6-diamino-purine, 5-bromodeoxyuridine or any other modified base which makes possible hybridization; at the sugar, for example, by the replacement of at least one deoxyribose by a polyamide (P. E. Nielsen et al., Science, 254, 1497-1500 (1991)); or at the phosphate group, for example, by replacement by esters chosen from diphosphate, alkyl- and arylphosphonate, and phosphorothioate esters. These modifications can be taken in combination.

The term “informational sequence” is understood to mean any orderly succession of monomers, the chemical nature of which and the order of which in a reference direction constitute data corresponding to that from natural nucleic acids.

A chemical polymer corresponds to the series of at least two identical or different monomer units. It preferably exhibits an average molecular mass of between 1 000 and 100 000. This polymer is preferably chosen from maleic anhydride homopolymers, copolymers based on maleic anhydride, copolymers based on N-vinylpyrrolidone, and polysaccharides. In particular, the polymer is chosen from poly(maleic anhydride-ethylene)s, poly(maleic anhydride-propylene)s, poly(maleic anhydride-methyl vinyl ether)s (MAMVE), N-vinylpyrrolidone-N-acryloxysuccinimide (NVP-NAS) and polysaccharides.

According to the invention, X is advantageously chosen from an aliphatic chain (CH₂)ₙ in which n is an integer equal to 6, an ethylene glycol, a tetra- or hexamethylene glycol, or a peptidyl residue comprising from 2 to 10 amino acids; and Y represents a polymer chosen from BSA, oligonucleotides composed of 18 to 22 mers, maleic anhydride homopolymers, copolymers based on maleic anhydride, copolymers based on N-vinylpyrrolidone, or polysaccharides.

In particular, the polymer is chosen from oligonucleotides composed of 20 mers and in particular the oligonucleotide known under the reference SEQ ID No. 2 and subsequently described in Example 5, poly(maleic anhydride-ethylene)s, poly(maleic anhydride-propylene)s, poly(maleic anhydride-methyl vinyl ether)s (MAMVE) and N-vinylpyrrolidone-N-acryloxysuccinimide (NVP-NAS). The polymer is advantageously coupled to at least one protein and/or one polypeptide and/or one oligonucleotide.

The novel abietane-derived conjugates can be used in a large number of analyses. By way of examples, said abietane-derived conjugates are used in diagnostic tests, such as immunological assays, or in tests using probe technology; in the monitoring of an infection, for example of a viral infection; or in the monitoring and/or the assay of chemicals and in particular of potentially polluting chemicals.

By way of examples, they can be used in an immunological diagnostic test as labels, as described beforehand for abietane derivatives, or in tests using probe technology for the detection and/or quantification of a nucleic acid fragment in a biological sample.

In probe technology, they can be used without distinction as capture and/or detection probes. Thus, in so-called sandwich technology, they can be used as a detection probe according to the following protocol: a capture probe is immobilized on a solid support, the capture phase thus formed is brought into contact with the target sequence of the sample and with an abietane-derived conjugate, which consists of an oligonucleotide coupled to an abietane derivative. The complex possibly formed is subsequently detected by addition of an antibody directed against the abietane derivative, said antibody being labeled by any appropriate label. They can also be used as a capture probe. In this respect, the abietane-derived conjugate, which consists of an oligonucleotide coupled to an abietane derivative, is immobilized on a solid support. The capture phase thus formed is brought into contact with the target sequence of the sample and the complex possibly formed is detected by a detection probe labeled by any appropriate label. The capture can be carried out directly or indirectly, that is to say either the conjugate is immobilized directly on the solid support or it is immobilized indirectly by coupling to an antibody directed against the abietane derivative attached beforehand to the solid support. Of course, the sandwich technique can be carried out in one stage or two stages. Likewise, the conjugates of the invention can be used for the detection of a target sequence in a sample both as capture probe and as detection probe. It is within the scope of a person skilled in the art to define the sequence and the length of the oligonucleotide of the conjugate as a function of the sequence of the target which is desired in the sample. It is also within the scope of a person skilled in the art to define capture and/or detection probes specific for the desired target.

Another subject matter of the present invention is thus a diagnostic reagent and a diagnostic composition comprising said reagent, the latter comprising:

a saturated or unsaturated abietane derivative corresponding to the preceding formula (I) in which Z is chosen from the group consisting of —COOR⁵, —CONR¹R², —COONR³R⁴, —COR⁶, —, —COOR⁵, —CHOHR⁷, —SR⁸, —OR⁸, —CN, —CH₂NO, —C H₂NS, —NCO, —NCS or —R¹R²CR⁹;

where R¹, R², R³ and R⁴, independently of one another, represent a hydrogen atom, an alkyl radical comprising from 1 to 10 carbon atoms, an aryl radical preferably comprising from 6 to 20 carbon atoms, which is optionally substituted; an alkene radical comprising from 7 to 10 carbon atoms; an alkyne radical comprising from 2 to 10 carbon atoms; or an optionally substituted aminoacyl or peptidyl radical, or R¹ and R² or R³ and R⁴ can together form a ring or a heterocycle; R⁵ represents a hydrogen atom, an alkyl radical comprising from 1 to 10 carbon atoms, an alkene radical comprising from 2 to 10 carbon atoms, an alkyne radical comprising from 2 to 10 carbon atoms or an optionally substituted aryl radical comprising from 6 to 20 carbon atoms; R⁶ represents a hydrogen atom, a halogen, an alkyl radical comprising from 1 to 10 carbon atoms, an alkene radical comprising from 2 to 10 carbon atoms, an alkyne radical comprising from 2 to 10 carbon atoms or an optionally substituted aryl radical comprising from 6 to 20 carbon atoms; R⁷ represents a hydrogen atom, an alkyl radical comprising from 1 to 10 carbon atoms, an alkene radical comprising from 2 to 10 carbon atoms or an alkyne radical comprising from 2 to 10 carbon atoms; R⁸ represents a hydrogen atom, an alkyl radical an alkyl radical comprising from 1 to 10 carbon atoms, an alkene radical comprising from 2 to 10 carbon atoms or an alkyne radical comprising from 2 to 10 carbon atoms; and R⁹ is chosen from —CN, —C H₂NO, —C H₂NS, —NCO and —NCS;

provided that, if said abietane derivative is an unsaturated derivative, Z does not represent a carboxylic acid radical, or

said derivative being coupled to a protein chosen from polypeptides, antigens and antibodies.

The invention also relates to the use of a reagent of the invention in a diagnostic test.

The conjugates of the invention can also be used to develop universal capture phases. For example, antibodies directed against an abietane derivative are immobilized, directly or indirectly, on a solid phase or support and the oligonucleotide of the conjugate of the invention is modified by attachment at one of its ends, preferably at the 5′-end, of an abietane derivative. The universal solid phase is composed of the solid phase/antibody directed against an abietane derivative assembly. Another subject matter of the invention is thus a device comprising, in addition to the solid phase or support, a polyclonal or monoclonal antibody immobilized directly or indirectly on the solid phase or support. The antibody is preferably a monoclonal antibody obtained according to known techniques and in particular according to the technique described in Example 10.

A conjugate of the invention comprising a chemical polymer can also be used as label or for developing a universal capture phase, as described above.

A conjugate of the invention can be used as immunogen, for the immunization of appropriate organisms, such as animals, or for the production of monoclonal or polyclonal antibodies. Another subject matter of the invention is thus a diagnostic reagent and a diagnostic composition additionally comprising said monoclonal or polyclonal antibodies and preferably monoclonal antibodies obtained according to known techniques, such as that described in Example 10. The invention also comprises the use of said reagent in a diagnostic test.

Furthermore, a conjugate of the invention can be used for the assay and/or the monitoring of chemicals, by a competition technique or by the so-called sandwich technique. Thus, another subject matter of the present invention is a composition additionally comprising a conjugate of the invention and a polyclonal or monoclonal antibody obtained by immunization of appropriate organisms using the abovementioned immunogen, preferably an anti-abietic acid monoclonal antibody obtained by immunization of an animal according to known techniques, such as that described subsequently in Example 10. The invention also relates to the use of a composition as defined above in the assay and/or the monitoring of chemicals.

The appended figure represents the signals obtained during the ELISA detection of the α-fetoprotein antigen, either by use of an anti-α-fetoprotein polyclonal antibody (round symbols) or by use of an MAMVE/abietic acid hydrazide/anti-α-fetoprotein polyclonal antibody conjugate of the invention (square symbols). The amounts of α-fetoprotein are represented on the abscissa in ng/ml and the OD values at 492 nm are represented on the ordinate.

### EXAMPLE 1

Synthesis of the N-hydroxysuccinimide Ester of Abietic Acid

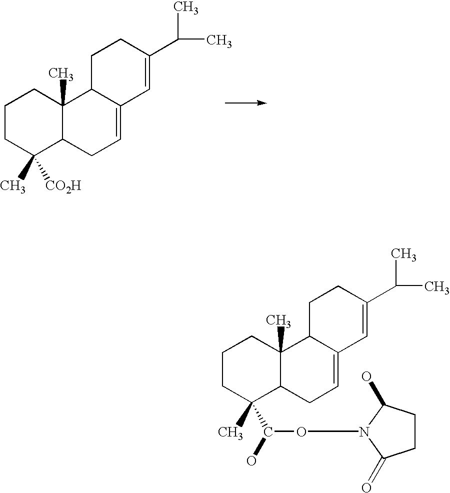

One gram of abietic acid (3.3 mmol, 1 equivalent (1 eq)) is dissolved in 40 ml of CH₂Cl₂ at a temperature of 0° C. in a 100 ml single-necked round-bottomed flask under a nitrogen atmosphere. 1.1 eq of N-hydroxysuccinimide are added. 1 eq of DDC is subsequently added dropwise, i.e. 0.825 ml of a 4 mol/l solution in anhydride CH₂cl₂. The reaction is carried out for 5 h at −20° C. and then the medium is diluted in 200 ml of ether and subsequently precipitated for 24 h at −20° C. After filtering through a sintered glass filter, the aqueous mother liquors are evaporated. Purification is carried out on 75 times the weight of silica, elution being carried out with a pentane/AcEtOH/CHCl₃ (65/25/10) mixture. 0.0998 gram of the ester C₂₄H₃₃O₄N is recovered, i.e. a yield of 75%.

C₂₄H₃₃O₄N Mw=399.54 g/mol IR: (cm^{31 1}) 2957 (CH₃, CH₂, CH); 1777 (C═O NHS ring); 1740 (C═O amide) NMR: H (ppm) 5.74 (s, CH), 5.3 (d, CH), 2.79 (s, CH₂, NHS), 1.29 (s, CH₃), 1.01 (d, CH₃), 0.98 (d, CH₃), 0.82 (s,CH₃). ¹³C (ppm) 173.5 (C═O), 169.2 (C═O), 145 (quater. C), 135.2 (quater. C), 122.4 (CH═), 120.4 (CH═), 25.6 (CH₂ of the NHS).

### EXAMPLE 2

Synthesis of an Activated glycyl-glycine Derivative of Abietic Acid

The synthesis is carried out according to the reaction scheme described below:

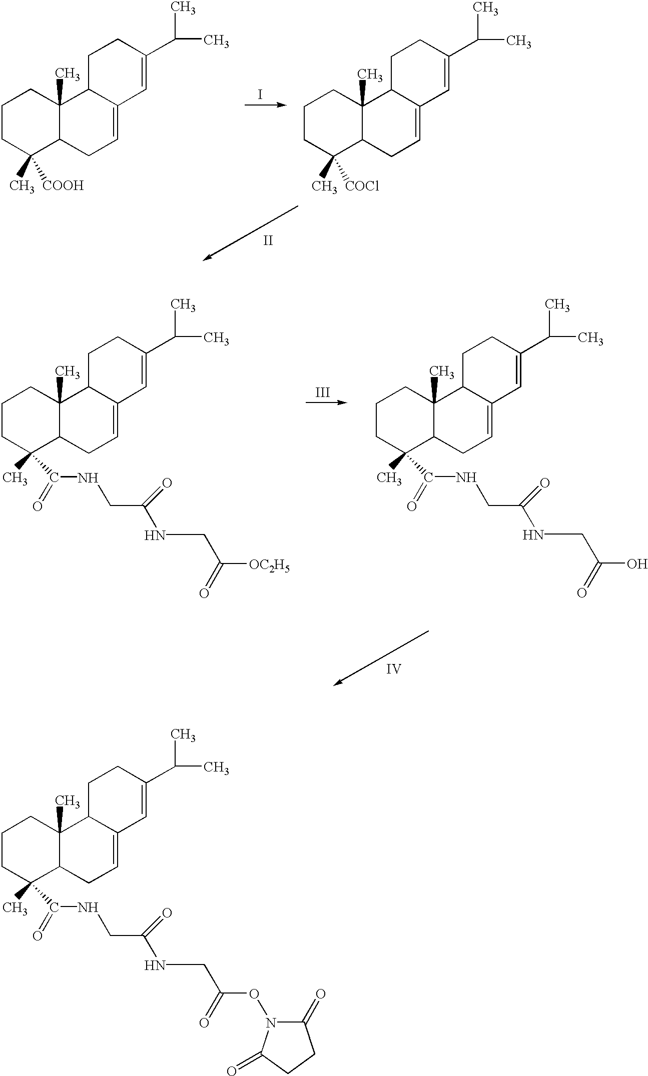

Synthesis of the acid chloride (i):

The abietic acid is recrystallized before synthesizing the chloride. 1.2 grams of abietic acid (3.97 mmol, 1 eq) are dissolved in 10 ml of anhydrous toluene in a 100 ml three-necked round-bottomed flask under a nitrogen atmosphere and are cooled to 0° C. 3 eq, i.e. 1 ml of oxalyl chloride, are added in solution in 3 ml of anhydrous toluene. After 15 min at 0° C., the reaction is left for 2 h at ambient temperature. The medium is piped into a single-necked round-bottomed flask and the toluene is removed under partial vacuum and then dried for 2 h under vacuum. The product can be used as is in the following stage. The complete disappearance of the acid is monitored by thin layer chromatography (pentane/AcEtOH/CHCl₃: 65/25/10). By infrared, the appearance of a band at 1775 cm⁻¹ shows the presence of acid chloride (O═C−Cl), which confirms the disappearance of the acid band at 1700 cm⁻¹ and of the OH band at 3000 cm⁻¹. The yield is quantitative.

Coupling of the ethyl ester of glycyl-glycine (ii):

1.95 grams of the ethyl ester of glycyl-glycine in the hydrochloride form (9.92 mmol, 1 eq) are dissolved in the presence of triethylamine (2 eq) in 30 ml of anhydrous dicloromethane (distilled over calcium hydride) in a 100 ml three-necked flask under nitrogen. The abietic acid chloride (9.92 mmol, 1 eq), dissolved in 15 ml of anhydrous dichloromethane, is slowly added to the preceding solution. The reaction is left for 7 h at ambient temperature. The amodium salts are precipitated from 200 ml of ether and then filtered off on a sintered glass filter. Washing operations are carried out with 0.1M HCl and then the organic phase is washed with a saturated NaCl solution before being dried over Na₂OS₄. After filtration, the solvent is removed under partial vacuum. The overall yield over the two stages is 83%.

Saponification of the ethyl ester of the glycyl-glycine (iii):

The saponification is carried out by dissolving approximately 2 grams of ester in 30 ml of ethanol and by slowly adding approximately 5 ml of NaOH. After 2 h, the ester has completely disappeared, which is confirmed by the Rf spots on silica (eluent 70/30: ether/acetone) (Rf ester=0.6 Rf acid=0). The medium is diluted in 250 ml of water and then washed three times with 50 ml of ether. After acidifying with 2N HCl, the compound is extracted by three times with 50 ml of ether. The ethereal phase is washed with saturated NaCl, dried over Na₂SO₄ and then filtered. The crude yield is 86%.

Activation of the glycyl-glycine derivative in the N-hydroxysuccinimide ester form (iv):

0.2645 gram of the acid obtained in the preceding stage (0.635 mmol, 1 eq) is dissolved in 40 ml of CH₂Cl₂ and then 0.111 gram of N-hydroxy-succinimide is added. The medium is cooled under a nitrogen atmosphere to 0° C. and 0.63 ml of DCC, as a 1M solution in CH₂Cl₂, is poured in in one step. The reaction is maintained at ambient temperature for 5 h and the dicyclohexylurea is precipitated at −20° C. for 24 h. The product (C₂₈H₃₉O₆N3) is filtered through 20 times the weight of silica, elution being carried out with an ether/acetone (80/20) mixture. The yield is 20%

MS (FAB+) C₂₈H₃₉O₆N₃ MH⁺ (calc.)=514.29169 g/mol MH⁺ (exp.)=514.29171 g/mol IR: (cm⁻¹) 1643 (C═0 amide II) 1740 (C═O amide) 1784, 1823 (C═O NHS) 2937 (CH₃, CH₂, CH) 3389 (NH amide) NMR: ¹H: (ppm) 0.79 (s, CH₃), 0. 95 (d, J=1.6 Hz, CH₃), 0.98 (d, J=1.4 Hz, CH₃), 1.25 (s, CH₃), 2.79 (CH₂, N-hydroxysuccinimide), 4.34 (d, J=5.6 Hz, CH₂), 5.3 (CH), 5.74 (s, CH). ¹³C: (ppm) [lacuna]

### EXAMPLE 3

Synthesis of a Hydrazide Derivative of Abietic Acid

The abietic acid is activated beforehand in the chloride form, as described in Example 3. 0.53 g of t-BOC hydrazide (4 mmol) is dissolved in 10 ml of anhydride toluene, to which are added 0.6 ml (2 eq) of triethylamine under a nitrogen atmosphere. 1 eq of acid chloride, in solution in toluene, are added dropwise at a temperature of 0° C. The reaction is left for 18 h at ambient temperature. After which the appearance of a precipitate is observed. The medium is acidified by addition of 4 ml of 1N HCl and the precipitate is filtered off on sintered glass. The medium is diluted in 50 ml of ether and, after drying over Na₂SO₄, the organic phase is evaporated under vacuum and pump dried. The product is filtered through 20 times the weight of silica, elution being carried out with a hexane/acetone (80/20) mixture. The product is isolated with a yield of 47%.

The t-BOC hydrazide is cleaved in anhydrous dioxane with a 4M solution of anhydrous HCl in anhydrous dioxane. After 20 h at ambient temperature, the precipitate appears. Bubbling with nitrogen makes it possible to entrain the remaining HCl. Evaporation on a rotary evaporator removes the acid and the dioxane. The residue is subsequently taken up in ether and then filtered through a sintered glass filter. The crude yield is 54%.

### EXAMPLE 4

Synthesis of a glycyl-glycine Hydrazide Derivative of Abietic Acid

1 ml of hydrazine hydrate (19 mmol, 21 eq), in solution in 5 ml of anhydrous dioxane, are vigorously stirred. 0.4777 g of ester obtained according to Example 3 (0.93 mmol, 1 eq), in solution in 45 ml of anhydrous dioxane, are added over 1 h 30. A precipitate appears after reacting for 50 min. After reacting for 4 h, the medium is precipitated from 250 ml of ether and filtration makes it possible to isolate a compound which is taken up in CHCl₃ and then dried under vacuum after concentrating. Dissolution in CHCl₃ is followed by reprecipitation from ether and 200 mg of product are isolated, i.e. a yield of 49%.

IR: (cm⁻¹ ) 1655 (C═0 amide II) 2932 (CH₃, CH₂, CH) 3319 (NH, NH₂ broad band)

### EXAMPLE 5

Coupling of the N-hydroxysuccinimide Ester of Abietic Acid to an Oligonucleotide Oligonucleotides are synthesized on a 394 automatic device from Applied Biosystems by using the chemistry of phosphoramidites according to the protocol of the manufacturer. In order to make possible the coupling of an oligonucleotide to the N-hydroxysuccinimide ester of abietic acid at a well determined position, reactive functional groups are introduced onto the oligonucleotide via binding arms which are compatible with the automatic synthesis, as disclosed in Patent FR 93 07093, the teaching of which is included by way of reference.

The following oligonucleotides were synthesized:

| SEQ ID No. | Nucleotide (*) | RT (**) |
|---|---|---|
| 1 | β | 19.54 |
| 2 | β | 18.45 |
| 3 | β | 19.85 |
| 4 | α | 23.21 |
| 5 | α | 20.04 |

| | |
|---|---|
| SEQ ID No. 1: | |
| ACTAAAAACT AGTAATGCAA AG | 22 mers |
| SEQ ID No. 2: | |
| ATGTCACGAG CAATTAAGCG | 20 mers |
| SEQ ID No. 3 | |
| ACTAAAAACT AGNAATGCAA AG | 22 mers |
| SEQ ID No. 4: | |
| ACCCCGAGAT TTACGTTATG T | 21 mers |
| SEQ ID No. 5: | |
| TTTTTTTTTT TTTTTTTTTT | 20 mers |

(*) the β nucleotides are natural nucleotides (the
glycoside bond is in the β anomeric form). The
α nucleotides are unnatural nucleotides (the
glycoside bond is in the α anomeric form) . The
oligonucleotides comprising α nucleotides were
prepared according to the technique disclosed in
Patent Application PCT W088/04301, the content of
which is incorporated by way of reference.
(**) RT represents the retention time in minutes
of the oligonucleotide under the conditions
disclosed in the abovementioned Patent
FR 93 07093.

200 μg of the synthetic oligonucleotide SEQ ID No. 2 are dissolved in 25 μl of 0.2M carbonate/0.15M NaCl buffer, pH=8.8. 500 μl of a 2.5 mg/ml solution of the ester C₂₄H₃₃O₄N in anhydrous DMSO are added to the solution of the oligonucleotide. The mixture is vigorously stirred and is then left for 4 h at 50° C. on a thermomixer. 10 μl [lacuna] 1M NH₄Cl, pH=6 are added at the end of the reaction. The medium is dried on a speed-vac and then taken up in water. Three extractions with butanol are carried out in order to remove the unreacted material. After lyophilization, the the oligonucleotide is taken up in doubly distilled water and analyzed by reverse phase HPLC on an RP3000 column with the following eluent: 0.1M TEAA0.1M TEAA/CH₃CN (50/50). The profile of the chromatogram shows the presence of unmodified oligonucleotide and an unresolved clump of more hydrophobic peaks comprising the oligonucleotide coupled to the ester. The yield, estimated by integrating the area of the peaks and regarding the molecular extinction coefficients as identical, is 40%.

### EXAMPLE 6

Coupling of the Ester C₂₈H₃₉O₆N₃ to an Oligonucleotide

The protocol is identical to that described in Example 5. A yield of isolated product of 20% is obtained after the extractions with butanol. Purification is carried out by HPLC.

### EXAMPLE 7

Coupling of the glycyl-glycine Hydrazide Derivative to the Polymer MAMVE

100 μl (1 mg) of a 10 mg/ml solution of MAMVE in anhydrous DMSO are reacted with 200 μl (0.2 mg) of abietic acid glycyl-glycine hydrazide in the presence of 20 μl of DIEA and 680 ml of DMSO. The reaction is left for 17 h at 37° C.

### EXAMPLE 8

Coupling of the Ester C₂₄H₃₃O₄N to BSA

20 mg of BSA (fraction 5—Sigma) are dissolved in 1 ml of 0.2M carbonate/0.5M NaCl buffer, pH=8.2. 960 μl of a 1 mg/ml solution of ester obtained according to Example 1 in anhydrous DMSO (i.e. 200 eq) are added to 40 μl of the above solution (1.212×10⁻⁸ mol). After vigorous stirring, the Eppendorf is stirred for 5 h at 37° C. on a thermomixer. The mixture is diluted in a large volume of water and then filtered on a Centricom (commercial name) PM30 at 7 RPM. The operation is repeated 3 times in order to remove the unreacted ester and the hydrolysis products, as well as to entrain the DMSO. The residue is dried on a speed-vac and then taken up in a volume of 1 ml of doubly distilled water. The conjugate is assayed after dilution by measuring the OD at 280 nm.

The conjugate obtained will be injected into mice in order to induce an immune response and the production of monoclonal antibodies, as described in Example 10.

### EXAMPLE 9

Coupling of the Ester C₂₈H₃₉O₆N₃ to BSA

The protocol is identical to that described in the preceding example. The coupling involves 40 or 80 eq of NHS esters. The conjugates obtained will be injected into mice in order to induce an immune response and the production of monoclonal antibodies, as described in the example which follows.

### EXAMPLE 10

Production of Anti-abietic Acid Monoclonal Antibodies

An antigen of abietic acid coupled to BSA as described respectively in Examples 10 and 11 is used as immunogen. Each of the antigens was injected into female mice of the BALB/C type and of the BALB/C BYJICO strain. The mice are immunized at intervals of 15 days using three injections by the intraperitoneal route associating respectively 50 μg of antigen and of complete Freund's adjuvant for the 1st injection and of incomplete Freund's adjuvant for the other injections. Fusion is carried out with the myeloma line SP2/O-AG14, according to the conventional technique described by Kölher and Milstein (Nature, 256, 495-497, 1975).

The cells were cultured using a base medium: Iscove's Modified Dulbecco Medium (IMDM) supplemented with sodium bicarbonate (3 024 mg/l) and with 10% of fetal calf serum (FCS), pH 6.7 to 7.3. The following additional reactants were added: insulin 4 mg/l, 2-mercaptoethanol (10 μM), ethanolamine (20 μM), penicillin (100 U/ml) and streptomycin (50 μg/ml). The heteroploid cells obtained were subcultured every two or three days and frozen in the IMDM medium supplemented with 10% of fetal calf serum (FCS) and 10% of dimethyl sulfoxide (DMSO sold by Sigma), first at −80° C. for 24 to 72 hours and then stored in liquid nitrogen at −180° C.

The cell concentration is 3.6×10⁶ cells per vial (2×10⁶ cells/ml).

The production of antibodies in vivo was carried out by intraperitoneal injection of the hybridoma lines obtained into female mice aged from 4 to 6 weeks of the BALB/C type of the BALB/C BYJICO strain.

### EXAMPLE 11

Screening of Anti-abietic Acid Antibodies

Screening was carried out by the indirect ELISA technique as follows:

NUNC Maxisorb polystyrene plates (sold by the company Polylabo Paul Block under the reference 4-39454) are sensitized with 100 μl of abietic acid coupled to an oligonucleotide (ODN) as described in Example 2, at 0.25 μg/ml of abietic acid-ODN in 3×PBS buffer (0.45M NaCl; 0.15M sodium phosphate; pH 7.0). The plates were incubated overnight at 22° C. or for one hour at 37° C. The plates were saturated with 100 μl of PBS buffer (50 mM phosphate and 150 mM NaCl, pH 7.2) to which 1% of dried milk extract (Régilait) had been added, for 1 hour at 37° C. 100 μl of antibodies, diluted in PBS-Tween 20 at 0.05%, were added and incubated for one hour at 37° C. 100 μl of anti-mouse total Ig immunoglobulins conjugated to alkaline phosphatase (Jackson Laboratories reference 115-055-062), diluted 2 000-fold in PBS buffer-1% BSA (phosphate buffer to saline-bovine serum albumin), were added and incubated for one hour at 37° C. The revelation was carried out by adding 100 μl of p-nitrophenyl phosphate ((pNPP), sold by bioMérieux, reference 60002990) at the concentration of 2 mg/ml in DEA-HCl (sold by bioMérieux, reference 60002989), pH 9.8, and incubating for 30 minutes for 37° C. The reaction was then blocked with 100 μl of 1N NaOH. Three washes were carried out between each step, with 300 μ of PBS-Tween 20 at 0.05%, and an additional wash was carried out in distilled water before adding the pNPP.

### EXAMPLE 12

Coupling of the Ester C₂₈H₃₉O₆N₃ to an Anti-α-fetoprotein Antibody (Anti-AFP)

The N-hydroxysuccinimide derivative is coupled to an anti-AFP monoclonal antibody in a mixture 0.1M sodium borate buffer, pH 9.2, containing 8% by volume of dimethyl sulfoxide. The antibody/derivative molar ratio is 1/45. The reaction medium is stirred for 4 hours at room temperature, before being dialyzed against PBS.

Immunological demonstration of the grafting of the abietic acid onto the antibody.

The wells of a NUNC MAXISORB microtitration plate are sensitized for two hours at 37° C. with a solution of the previously obtained antibody diluted to 10 μg/ml in a 50 mM carbonate buffer. After washing with PBS-0.5% Tween 20, solutions of peroxidase-labeled anti-abietic acid antibodies diluted in PBS-Tween containing 10% by volume of horse serum are incubated for one hour at 37° C. After washes, the enzymatic substrate, o-phenylenediamine, is added; the colorimetric reaction is stopped by adding 1N sulfuric acid. The intensity of the coloration is read at 492 nm on a bioMérieux Axia Micro-reader. The specific signals obtained are 2 500 milliabsorbances.

### EXAMPLE 13

Coupling of the glycyl-glycine Hydrazide Derivative to a Polymer and Grafting onto Anti-α-fetoprotein (AFP) Antibodies

10 μl (150 μg) of a 15 mg/ml solution of MAMVE in anhydrous DMSO are reacted with 10 μl (2 μg) of abietic acid glycyl-glycine hydrazide, in the presence of 980 μl of anhydrous DMSO. The mixture is vigorously stirred for 5 min, and then 15 μl of this medium are added to 1 ml of polyclonal anti-AFP antibodies at 0.5 mg/ml in a 50 mM Tris buffer, pH=9.2. The coupling is carried out overnight at 37° C.

### EXAMPLE 14

Coupling of the Abietic Acid and of a Rabbit Polyclonal Anti-α-fetoprotein Antibody to the MAMVE Copolymer

22 nmol of MANVE copolymer (molar mass 67 000 g/mol) and then 16 μmol of the hydrazide derivative derived from Example 3 are successively dissolved in 1 ml of dimethyl sulfoxide (DMSO). After stirring the reaction mixture for 20 minutes at room temperature, 15 μl of the solution are removed and added to 1 ml of the antibody solution at 0.5 g/l in a 50 mM Tris-HCl buffer, pH 9.2. The reaction medium is stirred at room temperature for 12 hours. The conjugates are conserved without modification at 40° C.

The grafting of the abietic acid and of the polyclonal antibody onto the MAMVE copolymer is controlled using an immunological test as follows:

A mouse monoclonal antibody directed against the abietic acid obtained according to Example 10 is diluted to the concentration of 10 μg/ml in a 50 mM carbonate buffer. The wells of a NUNC MAXISORB microtitration plate are sensitized with this antibody for two hours at 37° C. After washes with PBS (Phosphate Buffer Saline)-0.5% Tween 20, solutions of MAMVE/rabbit polyclonal anti-α-fetoprotein antibody/ambietic acid hydrazide derivative complexes diluted in PBS-Tween containing 10% by volume of horse serum are incubated for one hour at 37° C. There are two possibilities for detection at this point in the experiment:

Detection of the attachment of the abietic acid hydrazide derivative

After three washes with PBS-Tween, incubation is carried out for one hour at 37° C. with a monoclonal anti-abietic acid antibody conjugated to peroxidase, diluted 1 500-fold in PBS-Tween containing 10% by volume of horse serum. After washes, the enzymatic substrate, o-phenylenediamine, is added; the colorimetric reaction is stopped by adding 1N sulfuric acid. The intensity of the coloration is read at 492 nm on a bioMérieux Axia Micro-reader. The optical density values obtained are 2 500 milliabsorbances for the samples, and 170 for the negative control, thus proving that the abietic acid hydrazide derivative had indeed attached to the polymer.

Detection of the attachment of the polyclonal antibody to the MAMVE copolymer.

After three washes with PBS-Tween, incubation is carried out for one hour at 37° C. with an anti-goatantibody antibody conjugated to peroxidase, diluted 3 000-fold in PBS-Tween containing 10% by volume of horse serum. After washes, the enzymatic substrate, o-phenylenediamine, is added; the colorimetric reaction is stopped by adding 1N sulfuric acid. The intensity of the coloration is read at 492 nm on a bioMérieux Axia Micro-reader. The optical density values read are 2 500 milliabsorbances for the samples, and 25 for the negative control, thus proving that the polyclonal anti-AFP antibody has indeed attached to the polymer.

This example shows that polymers carrying abietic acid can be detected using a sandwich technique.

### EXAMPLE 15

Amplification of the Signal for Detecting the α-fetoprotein Antigen, by the MAMVE/abietic Acid Hydrazide Derivative/polyclonal Anti-α-fetoprotein Antibody Mixed Complexes

A mouse monoclonal antibody directed against α-fetoprotein (AFP) is diluted to the concentration of 10 μg/ml in a 50 mM carbonate buffer. The wells of a NUNC MAXISORP microtitration plate are sensitized for two hours at 37° C. with this antibody. After washing with PBS-0.5% Tween 20, solutions of AFP antigen diluted in PBS-Tween containing 10% by volume of horse serum are incubated for one hour at 370+ C. At this stage, two detection methods can be envisaged; either detection in a nonamplified way, or detection using the mixed complex previously obtained in order to increase the signal level.

Simple detection of the AFP antigen

After three washes with PBS-Tween, the polyclonal anti-AFP antibody conjugated to peroxidase, diluted 3 000-fold in PBS-Tween containing 10% by volume of horse serum, is incubated for one hour at 37° C. After washes, the enzymatic substrate, o-phenylenediamine, is added; the colorimetric reaction is stopped by adding lN sulfuric acid. The intensity of the coloration is read at 492 nm on a bioMérieux Axia Micro-reader. The optical density (OD) values, read at 492 nm on the bioMérieux Axia Micro-reader, for the samples are given in the appended figure.

Detection amplified by the AFP antigen polymer

After three washes with PBS-Tween, the polymer/abietic acid hydrazide/polyclonal anti-AFP antibody mixed conjugate, diluted 50-fold in PBS-Tween containing 10% by volume of horse serum, is incubated for one hour at 37° C. After washes, a peroxidase-labeled anti-abietic acid antibody is incubated for one hour at 37° C. After washes, the enzymatic substrate, o-phenylenediamine, is added. The colorimetric reaction is stopped by adding 1M sulfuric acid. The intensity of the coloration is read at 492 nm on a bioMérieux Axia Micro-reader. The OD values, read at 492 nm on the bioMérieux Axia Micro-reader, for the samples are given in the appended figure.

The results show that the use of polymer makes it possible to amplify the detection signals when using an ELISA technique.

### EXAMPLE 16

Competition Assay with Natural Hormones

A competition ELISA assay was carried out according to the technique described in patent FR 93 07093 cited above, using the natural hormones T3, T4, progesterone, testosterone and estradiol. Although steroid hormones have a similar structure to abietic acid, the antibodies developed must not exhibit crossreactions with these hormones.

The antigen phase was obtained as described in Example 11, and then increasing concentrations of hormone dissolved in a 5 μg/ml solution of anti-abietic acid antibody in PBS-Tween containing 10% by volume of horse serum are incubated for one hour at 37° C. After washes with PBS-Tween, an anti-mouse antibody antibody coupled to peroxidase, in solution in PBS-Tween-horse [lacuna] is incubated for one hour at 37° C. After washes, the enzymatic substrate, o-phenylenediamine, is added. The colorimetric reaction is stopped by adding 1N sulfuric acid. The intensity of the coloration is read at 492 nm on a bioMérieux Axia Micro-reader.

The results show that there is no inhibition with the steroid hormones such as estradiol, testosterone and progesterone. On the other hand, a crossreaction is observed with the thyroid hormones T3 and T4. 25% inhibition is obtained for a concentration of 0.33 μmol/ml of hormone, which is well above the physiological concentrations of total (i.e. free and bound) hormone, which are, respectively, 2-4 nmol/l for T3 and 50-100 nmol/l for T4. These results make it possible to conclude that the anti-abietic acid antibodies are very specific and can be used in the analytical domain without any risk of crossreaction.

### APPENDIX

3. Derivative according to claim 1 and 2, in which —COONR³R⁴ represents an N-hydroxysuccinimide ester, —COR⁶ represents an acid chloride, —CONR¹R² represents an N-substituted amide group in which R¹ and R², independently of one another, represents [sic] a hydrogen atom, a polyethylene glycol radical or an optionally substituted peptidyl radical comprising from 2 to 6 aminoacyl residues and —COOR⁵ is a polyethylene glycol ester.

9. Conjugate according to an, one of claim 6, 7 or 8 in which X is chosen from (CH₂)₆, an ethylene glycol, a sic tetra- or hexaethyl [sic] glycol, or a peptidyl radical comprising from 2 to 10 aminoacyl residues and Y represents a polymer chosen from BSA,

12. Process for the production of monoclonal or polyclonal antibodies, according to which an appropriate organism is immunized, according to known techniques, with a conjugate as defined in claims 6 to 11.

15. Use of a reagent as defined according to claims 13 and/or 14 in a diagnostic test.

16. Diagnostic composition, additionally comprising a reagent as defined according to claims 13 and/or 14

17. Reagent, additionally comprising a conjugate as defined according to claims 6 to 11

18. Use of a reagent as defined according to claims 14 and/or 17 in a diagnostic test.

19. Diagnostic composition, additionally comprising a reagent as defined according to claims 11 and/or 17.

21. Composition for the assay and/or the monitoring of chemicals, additionally comprising a conjugate as defined in claims 6 to 11 and an antibody obtained according to the bus of claim 12.

22. Composition according to claim 21 in which said conjugate is defined in one of claim [sic] 10 and 11.

23. Use of a composition according to claims 21 and 22 in a test for the assay and/or the monitoring of chemicals.

1. Saturated or unsaturated abietane derivative of general formula (I) 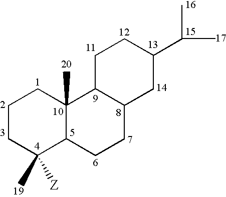
in which Z is chosen from the group consisting of —COOR⁵, —CONR¹R², —COONR³R⁴, —COR⁶, —COOR⁵ [sic], —CHOHR⁷, —SR⁸, —OR⁸, —CN, —CH₂NO, —CH₂NS, —NCO, —NCS or —R¹R²CR⁹;
where R¹, R², R³ and R⁴, independently of one another, represent a hydrogen atom, an alkyl radical comprising from 1 to 10 carbon atoms, an aryl radical preferably comprising from 6 to 20 carbon atoms, which is optionally substituted; an alkene [sic] radical comprising from 7 to 10 carbon atoms; an alkyne [sic] radical comprising from 2 to 10 carbon atoms; or an optionally substituted aminoacyl or peptidyl radical, or R¹ and R² or R³ and R⁴ can together form a ring or a heterocycle; R⁵ represents a hydrogen atom, an alkyl radical comprising from 1 to 10 carbon atoms, an alkene [sic] radical comprising from 2 to 10 carbon atoms, an alkyne [sic] radical comprising from 2 to 10 carbon atoms or an optionally substituted aryl radical comprising from 6 to 20 carbon atoms; R⁶ represents a hydrogen atom, hydrogen [sic], a halogen, an alkyl radical comprising from 1 to 10 carbon atoms, an alkene [sic] radical comprising from 2 to 10 carbon atoms, an alkyne [sic] radical comprising from 2 to 10 carbon atoms or an optionally substituted aryl radical comprising from 6 to 20 carbon atoms; R⁷ represents a hydrogen atom, an alkyl radical comprising from 1 to 10 carbon atoms, an alkene [sic] radical comprising from 2 to 10 carbon atoms or an alkyne [sic] radical comprising from 2 to 10 carbon atoms; R⁸ represents a hydrogen atom, an alkyl radical an alkyl radical [sic] comprising from 1 to 10 carbon atoms, an alkene [sic] radical comprising from 2 to 10 carbon atoms or an alkyne [sic] radical comprising from 2 to 10 carbon atoms; and R9 [sic] is chosen from —CN, —CH₂NO, —CH₂NS, —NCO and —NCS;
provided that
(a) if said abietane derivative is a saturated derivative:
Z does not represent any one of the following radicals: —COOH, —NCO, —CONH² [sic], —CN, N-benzylamide, N-isopropylamide, N-cyclohexylamide, N-cyclopentylamide, N-α-phenyl-ethylamide, N,N-dibenzylamide, N-methyl-N-cyclohexylamide, N-methyl-N-phenylamide, N-phenyl-N-benzylamide, anilide and —CONH[—(CH₂)ₘ—C₆H₅₋ₙX′ₙ], where m is equal to 0 or 1, n is equal to 1, 2 or 3, and X′ represents a halogen atom, a lower alkyl group of methyl or ethyl type, a haloalkyl group, a hydroxyl group, a lower alkoxy group of methoxy or alkoxy [sic] type, a nitro group, a carbonyl [sic] group, an alkoxycarbonyl group and a methyl group, and
(b) if said abietane derivative is an unsaturated derivative:
Z does not represent any one of the following radicals: —COOH, N-isopropylamide, N-methyl-N-cyclohexylamide, N-cyclohexylamide, N-decyl-amide, N-dodecylamide, N-pentadecylamide, N-allylamide, N,N-diallylamide, N-cyclo-heptylamide, N-cyclopentylamide, N-benzylamide, N-α-phenylethylamide, N-α-phenylpropylamide, N,N-dibenzylamide, N-β-phenylethylamide, N-ethyl-N-benzylamide, N-methyl-N-phenylamide, anilide and —CONH[—(CH₂)ₘ—C₆H₅₋ₙX′ₙ], where m is equal to 0 or 1, n is equal to 1, 2 or 3, and X′ represents a halogen atom, a lower alkyl group of methyl or ethyl type, a haloalkyl group, a hydroxyl group, a lower alkoxy group of methoxy or ethoxy type, a nitro group, a carbonyl [sic] group, an alkoxycarbonyl group and a methyl group,
if Z represents —COOR⁵, R⁵ represents neither H nor a methyl, ethyl or benzyl radical,
if Z represents —CONR¹R² and if one of R¹ and R² represents H, the other of R¹ and R² does not represent H, and if one of R¹ and R² represents the ethyl radical, the other of R¹ and R² does not represent the ethyl radical,
if Z represents —COR⁶ or —CHOHR⁷, R⁶ and R⁷ do not represent H.

2. Derivative according to claim 1, in which the alkyl radical comprises from 1 to 6 carbon atoms and the aryl radical comprises from 6 to 14 carbon atoms.

3. Derivative according to claims 1 and 2, in which —COONR³R⁴ represents an N-hydroxysuccinimide ester, —COR⁶ represents an acid chloride, —CONR¹R² represents an N-substituted amide group in which R¹ and R², independently of one another, represents [sic] a hydrogen atom, a polyethylene glycol radical or an optionally substituted peptidyl radical comprising from 2 to 6 aminoacyl residues and —COOR⁵ is a polyethylene glycol ester.

4. Derivative according to claim 3, in which R¹ and R², independently of one another, represents [sic] a hydrogen atom, a tetraethylene glycol radical, a hexaethylene glycol radical or a glycyl-glycine radical and —COOR⁵ is chosen from tetraethylene glycol and hexaethylene glycol esters.

5. Derivative according to claim 4, in which the glycyl-glycine radical is substituted by N-hydroxy-succinimide.

6. Abietane-derived conjugate according to the formula (II) 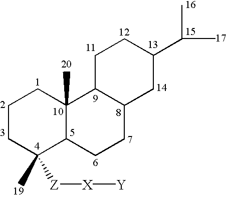
in which Z is chosen from the group consisting of —COOR⁵, —CONR¹R², —COONR³R⁴, —COR⁶, —COOR⁵ [sic], —CHOHR⁷, —SR⁸, —OR⁸, —CN, —CH₂NO, —CH₂NS, —NCO, —NCS or —R¹R²CR⁹;
in which R¹, R², R³ and R⁴ independently of one another, represent a hydrogen atom, an alkyl radical comprising from 1 to 10 carbon atoms, an aryl radical comprising from 6 to 20 carbon atoms, which is optionally substituted; an alkene [sic] radical comprising from 7 to 10 carbon atoms; an alkyne [sic] radical comprising from 2 to 10 carbon atoms; or an optionally substituted aminoacyl or peptidyl radical, or R¹ and R² or R³ and R⁴ can together form a ring or a heterocycle; R⁵ represents a hydrogen atom, an alkyl radical comprising from 1 to 10 carbon atoms, an alkene [sic] radical comprising from 2 to 10 carbon atoms, an alkyne [sic] radical comprising from 2 to 10 carbon atoms or an optionally substituted aryl radical comprising from 6 to 20 carbon atoms; R⁶ represents a hydrogen atom, a halogen, an alkyl radical comprising from 1 to 10 carbon atoms, an alkene [sic] radical comprising from 2 to 10 carbon atoms, an alkyne [sic] radical comprising from 2 to 10 carbon atoms or an optionally substituted aryl radical comprising from 6 to 20 carbon atoms; R⁷ represents a hydrogen atom, an alkyl radical comprising from 1 to 10 carbon atoms, an alkene [sic] radical comprising from 2 to 10 carbon atoms or an alkyne [sic] radical comprising from 2 to 10 carbon atoms; R⁸ represents a hydrogen atom, an alkyl radical comprising from 1 to 10 carbon atoms, an alkene [sic] radical comprising from 2 to 10 carbon atoms or an alkyne [sic] radical comprising from 2 to 10 carbon atoms; and R⁹ is chosen from —CN, —CH₂NO, —CH₂NS, —NCO and —NCS;
X is chosen from an aliphatic chain (CH₂)n in which n is an integer between 0 and 10, an ethylene glycol or a polyethylene glycol, or an aminoacyl or peptidyl residue;
Y represents a polymer chosen from proteins, polypeptides, oligonucleotides or polynucleotides, and chemical polymers;
provided that, if said conjugate comprises an unsaturated abietane derivative, Y is not BSA.

7. Conjugate according to claim 6, in which —COONR³R⁴ represents an N-hydroxysuccinimide ester, —COR⁶ represents an acid chloride and —CONR¹R² represents an N-substituted amide group in which R¹ and R², independently of one another, represents [sic] a hydrogen atom or an optionally substituted peptidyl radical comprising from 2 to 6 aminoacyl residues.

8. Conjugate according to claim 6, in which the peptidyl radical is a glycyl-glycine radical and advantageously the glycyl-glycine radical is substituted by N-hydroxysuccinimide.

9. Conjugate according to any one of claim 6, 7 or 8, in which X is chosen from (CH₂)₆, an ethylene glycol, aa [sic] tetra- or hexaethyl [sic] glycol, or a peptidyl radical comprising from 2 to 10 aminoacyl residues and Y represents a polymer chosen from BSA, oligonucleotides composed of 18 to 22 mers, maleic anhydride homopolymers, copolymers based on maleic anhydride, copolymers based on N-vinylpyrrolidone and polysaccharides.

10. Conjugate according to claim 9, in which the polymer is chosen from oligonucleotides composed of 20 mers and advantageously the oligonucleotide SEQ ID No. 2, poly(maleic anhydride-ethylene)s, poly(maleic anhydride-propylene)s, poly(maleic anhydride-methyl vinyl ether)s (MAMVE) and N-vinylpyrrolidone-N-acryloxysuccinimide [sic] (NVP-NAS).

11. Conjugate according to claim 10, in which the polymer is coupled to at least one protein and/or one polypeptide and/or one oligonucleotide.

12. Process for the production of monoclonal or polyclonal antibodies, according to which an appropriate organism is immunized, according to known techniques, with a conjugate as defined in claims 6 to 11.

13. Reagent, additionally comprising a saturated or unsaturated abietane derivative corresponding to the formula (I) 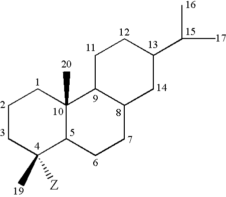
in which Z is chosen from the group consisting of —COOR⁵ —CONR¹R², —COONR³R⁴, —COR⁶, —COOR⁵ [sic], —CHOHR⁷, —SR⁸, —OR⁸, —CN, —CH₂NO, —CH₂NS, —NCO, —NCS or —R¹R²CR⁹;
where R¹, R², R³ and R⁴, independently of one another, represent a hydrogen atom, an alkyl radical comprising from 1 to 10 carbon atoms, an aryl radical preferably comprising from 6 to 20 carbon atoms, which is optionally substituted; an alkene [sic] radical comprising from 7 to 10 carbon atoms; an alkyne [sic] radical comprising from 2 to 10 carbon atoms; or an optionally substituted aminoacyl or peptidyl radical, or R¹ and R² or R³ and R⁴ can together form a ring or a heterocycle; R⁵ represents a hydrogen atom, an alkyl radical comprising from 1 to 10 carbon atoms, an alkene [sic] radical comprising from 2 to 10 carbon atoms, an alkyne [sic] radical comprising from 2 to 10 carbon atoms or an optionally substituted aryl radical comprising from 6 to 20 carbon atoms; R⁶ represents a hydrogen atom, a halogen, an alkyl radical comprising from 1 to 10 carbon atoms, an alkene [sic] radical comprising from 2 to 10 carbon atoms, an alkyne [sic] radical comprising from 2 to 10 carbon atoms or an optionally substituted aryl radical comprising from 6 to 20 carbon atoms; R⁷ represents a hydrogen atom, an alkyl radical comprising from 1 to 10 carbon atoms, an alkene [sic] radical comprising from 2 to 10 carbon atoms or an alkyne [sic] radical comprising from 2 to 10 carbon atoms; R⁸ represents a hydrogen atom, an alkyl radical an alkyl radical [sic] comprising from 1 to 10 carbon atoms, an alkene [sic] radical comprising from 2 to 10 carbon atoms or an alkyne [sic] radical comprising from 2 to 10 carbon atoms; and R⁹ is chosen from —CN, —CH₂NO, —CH₂NS, —NCO and —NCS;
provided that, if said abietane derivative is an unsaturated derivative, Z does not represent a carboxylic acid radical,
said derivative being coupled to a protein chosen from polypeptides, antigens and antibodies.

14. Reagent, additionally comprising a monoclonal or polyclonal antibody obtained according to the process of claim 12, said antibody being immobilized on a solid support and/or labeled by any appropriate label.

15. Use of a reagent as defined according to claims 13 and/or 14 in a diagnostic test.

16. Diagnostic composition, additionally comprising a reagent as defined according to claims 13 and/or 14.

17. Reagent, additionally comprising a conjugate as defined according to claims 6 to 11.

18. Use of a reagent as defined according to claims 14 and/or 17 in a diagnostic test.

19. Diagnostic composition, additionally comprising a reagent as defined according to claims 14 and/or 17.

20. Device comprising, in addition to a solid support, a monoclonal or polyclonal antibody obtained according to the process of claim 12 immobilized directly or indirectly on said solid support.

21. Composition for the assay and/or the monitoring of chemicals, additionally comprising a conjugate as defined in claims 6 to 11 and an antibody obtained according to the process of claim 12.

22. Composition according to claim 21, in which said conjugate is defined in one of claim [sic] 10 and 11.

23. Use of a composition according to claims 21 and 22 in a test for the assay and/or the monitoring of chemicals.


SEQUENCE LISTING (5 sequences)

SEQ ID NO: 1 - DNA, 22 bp, Artificial Sequence
  Synthetic Oligonucleotide
actaaaaactagtaatgcaaag

SEQ ID NO: 2 - DNA, 20 bp, Artificial Sequence
  Synthetic Oligonucleotide
atgtcacgagcaattaagcg

SEQ ID NO: 3 - DNA, 22 bp, Artificial Sequence
  Synthetic Oligonucleotide
actaaaaactagnaatgcaaag

SEQ ID NO: 4 - DNA, 21 bp, Artificial Sequence
  Synthetic Oligonucleotide
accccgagatttacgttatgt

SEQ ID NO: 5 - DNA, 20 bp, Artificial Sequence
  Synthetic Oligonucleotide
tttttttttttttttttttt